SEQUENCE LISTING (14 sequences)

SEQ ID NO: 1 - protein, 49 aa, synthetic construct
FYAVRRGRKTGVFLTWNECRAQVDRFPAARFKKFATEDEAWAFVRKSAS

SEQ ID NO: 2 - DNA, 147 bp, synthetic construct
  source 1..147: mol_type = other DNA
ttctatgccgtgaggaggggccgcaagaccggggtctttctgacctggaatgagtgcagagcacaggtggacagatttcctgctgccagatttaagaagtttgccacagaggatgaggcctgggcctttgtcaggaaatcagcaagc

SEQ ID NO: 3 - protein, 50 aa, synthetic construct
MFYAVRRGRKTGVFLTWNECRAQVDRFPAARFKKFATEDEAWAFVRKSAS

SEQ ID NO: 4 - DNA, 150 bp, synthetic construct
  source 1..150: mol_type = other DNA
atgttctatgccgtgaggaggggccgcaagaccggggtctttctgacctggaatgagtgcagagcacaggtggacagatttcctgctgccagatttaagaagtttgccacagaggatgaggcctgggcctttgtcaggaaatcagcaagc

SEQ ID NO: 5 - protein, 50 aa, synthetic construct
VFYAVRRGRKTGVFLTWNECRAQVDRFPAARFKKFATEDEAWAFVRKSAS

SEQ ID NO: 6 - DNA, 150 bp, synthetic construct
  source 1..150: mol_type = other DNA
gtgttctatgccgtgaggaggggccgcaagaccggggtctttctgacctggaatgagtgcagagcacaggtggacagatttcctgctgccagatttaagaagtttgccacagaggatgaggcctgggcctttgtcaggaaatcagcaagc

SEQ ID NO: 7 - protein, 236 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 8 - protein, 711 aa, synthetic construct
ATGGTGAGCAAGGGCGAGGAGGATAACATGGCCTCTCTCCCAGCGACACATGAGTTACACATCTTTGGCTCCATCAACGGTGTGGACTTTGACATGGTGGGTCAGGGCACCGGCAATCCAAATGATGGTTATGAGGAGTTAAACCTGAAGTCCACCAAGGGTGACCTCCAGTTCTCCCCCTGGATTCTGGTCCCTCATATCGGGTATGGCTTCCATCAGTACCTGCCCTACCCTGACGGGATGTCGCCTTTCCAGGCCGCCATGGTAGATGGCTCCGGCTACCAAGTCCATCGCACAATGCAGTTTGAAGATGGTGCCTCCCTTACTGTTAACTACCGCTACACCTACGAGGGAAGCCACATCAAAGGAGAGGCCCAGGTGAAGGGGACTGGTTTCCCTGCTGACGGTCCTGTGATGACCAACTCGCTGACCGCTGCGGACTGGTGCAGGTCGAAGAAGACTTACCCCAACGACAAAACCATCATCAGTACCTTTAAGTGGAGTTACACCACTGGAAATGGCAAGCGCTACCGGAGCACTGCGCGGACCACCTACACCTTTGCCAAGCCAATGGCGGCTAACTATCTGAAGAACCAGCCGATGTACGTGTTCCGTAAGACGGAGCTCAAGCACTCCAAGACCGAGCTCAACTTCAAGGAGTGGCAAAAGGCCTTTACCGATGTGATGGGCATGGACGAGCTGTACAAGTAA

SEQ ID NO: 9 - protein, 10 aa, synthetic construct
VDGTAGPGSA

SEQ ID NO: 10 - DNA, 30 bp, synthetic construct
  source 1..30: mol_type = other DNA
gtggacggaacagcgggcccgggatctgcc

SEQ ID NO: 11 - protein, 22 aa, synthetic construct
VDGTAGPGSTGVTGKLDPPVAT

SEQ ID NO: 12 - DNA, 66 bp, synthetic construct
  source 1..66: mol_type = other DNA
gtggacggcaccgctggcccgggatcgaccggagttacaggtaagctggatccaccggtcgccacc

SEQ ID NO: 13 - protein, 428 aa, synthetic construct
MFYAVRRGRKTGVFLTWNECRAQVDRFPAARFKKFATEDEAWAFVRKSASVDGTAGPGSAVFYAVRRGRKTGVFLTWNECRAQVDRFPAARFKKFATEDEAWAFVRKSASVDGTAGPGSAVFYAVRRGRKTGVFLTWNECRAQVDRFPAARFKKFATEDEAWAFVRKSASVDGTAGPGSTGVTGKLDPPVATMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 14 - DNA, 1287 bp, synthetic construct
  source 1..1287: mol_type = other DNA
atgttctatgccgtgaggaggggccgcaagaccggggtctttctgacctggaatgagtgcagagcacaggtggacagatttcctgctgccagatttaagaagtttgccacagaggatgaggcctgggcctttgtcaggaaatcagcaagcgtggacggaacagcgggcccgggatctgccgtgttctatgccgtgaggaggggccgcaagaccggggtctttctgacctggaatgagtgcagagcacaggtggacagatttcctgctgccagatttaagaagtttgccacagaggatgaggcctgggcctttgtcaggaaatcagcaagcgtggacggcaccgctggcccgggatctgccgtgttctatgccgtgaggaggggccgcaagaccggggtctttctgacctggaatgagtgcagagcacaggtggacagatttcctgctgccagatttaagaagtttgccacagaggatgaggcctgggcctttgtcaggaaatcagcaagcgtggacggcaccgctggcccgggatcgaccggagttacaggtaagctggatccaccggtcgccaccatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtaa

(19) United States
(12) Patent Application Publication
(10) Pub. No.: US 2026/0234584 A1
(43) Pub. Date: Aug. 13, 2026

(54) R-LOOP SENSOR

(71) Applicants: FUNDAÇÃO GIMM - GULBENKIAN INSTITUTE FOR MOLECULAR MEDICINE, Lisboa (PT); FACULDADE DE MEDICINA DA UNIVERSIDADE DE LISBOA, Lisboa (PT)

(72) Inventors: Sérgio Fernandes de Almeida, Lisboa (PT); Robert Manfred Martin, Lisboa (PT)

(21) Appl. No.: 19/160,422

(22) PCT Filed: Mar. 1, 2024
(86) PCT No.: PCT/EP2024/055481

§ 371 Date: Aug. 27, 2025

(30) Foreign Application Priority Data
Mar. 2, 2023 (PT) PT118540

(51) Int. Cl. C12N 9/22; C07K 14/435; C12Q 1/6897

(52) U.S. Cl. CPC C12N 9/22; C07K 14/43595; C12Q 1/6897; C12Y 301/26004; C07K 2319/60; C07K 2319/61

(57) ABSTRACT

This invention relates to a method of detecting an R-loop in a cell comprising culturing an isolated cell expressing a DNA:RNA hybrid-binding fusion protein that comprises (a) two or more hybrid binding domains (HBDs); and (b) a detectable reporter domain, such that the fusion protein binds to R-loops in the cell. The distribution of the fusion protein within the cell is then determined. An accumulation of fusion protein is indicative of an R-loop in the cell. Also provided are DNA:RNA hybrid-binding fusion proteins, encoding nucleic acids, and methods of screening for compounds that modulate R-loops.

### FIELD

The invention relates to agents that selectively bind to R-loops in cells, and methods of use thereof.

### BACKGROUND

R-loops are abundant non-canonical nucleic acid structures composed of a double-stranded DNA:RNA hybrid and a displaced DNA strand. R-loops are usually formed during transcription, when the nascent RNA molecule hybridizes with the template DNA strand, preventing the non-template strand from annealing to the template strand.

Most described physiological roles of R-loops relate to their impact on protein-coding gene transcription by RNA polymerase II. For example, R-loops may both activate and silence gene expression, by regulating the recruitment of transcription factors and by facilitating epigenetic and chromatin remodeling (Niehrs C. Nat Rev Mol Cell Biol. 2020 March; 21 (3): 167-178). R-loops are also implicated as modulators of genome instability and DNA damage (Marnef A. Nat Cell Biol. 2021 April; 23 (4): 305-313). Consequently, R-loops are thought to contribute to in the pathogenesis of various cancers and degenerative diseases, such as amyotrophic lateral sclerosis (ALS), frontotemporal dementia (FTD), polyglutamine-associated ataxias, spinocerebellar ataxias, Huntington's disease, Friedreich's ataxia and Aicardi-Goutières syndrome (Khan E S. Genes. 2022; 13(12): 2181):

The anti-DNA:RNA hybrid antibody S9.6 is canonically used to identify R-loops. However, S9.6 is known to bind double-stranded RNA (dsRNA) molecules and is unsuitable for use in live cells (Bou-Nader C. Nature Communications. 2022 March; 13 (1): 1641; Smolka J A. J Cell Biol. 2021 Jun. 7; 220 (6): e202004079).

Other commonly used methods to map and quantify R-loops exploit the RNase H1 enzyme. RNase H1 degrades the RNA moiety of R-loops, thereby restoring the double-stranded conformation of the two DNA strands (Cerritelli S M. Methods Mol Biol. 2022; 2528:91-114). Its specificity towards DNA:RNA hybrids is conferred by a hybrid binding domain (HBD). RNase H1 overexpression has served to inspect R-loops and the many roles R-loops play in processes such as gene expression regulation, class-switch recombination or genome damage and repair.

Catalytically dead RNase H1 has been employed as R-loop detection tools for imaging fixed cells and immunoprecipitation-based assays (Wang K. Sci Adv. 2021 Feb. 17; 7 (8): eabe3516; Crossley M P. J Cell Biol. 2021 Sep. 6; 220 (9): e202101092). Similarly, a single, fluorescently tagged RNase H1 HBD was previously used to detect R-loops in live cells (Silva, S. Methods Mol. Biol. 2022 Jun. 16 2528). However, like S9.6, the use of catalytically dead RNase H1 or isolated RNase HBDs in live cell imaging has proved problematic, in view of poor antigen specificity and low image quality (e.g., low signal-to-noise ratio).

Access to tools capable of specifically and sensitively detecting R-loops and for measuring the kinetics of R-loop formation and resolution would therefore be advantageous in the study of various diseases, such as cancers and degenerative diseases.

### SUMMARY

The present inventors have unexpectedly found that the expression of an DNA:RNA hybrid-binding fusion protein that comprises two or more hybrid binding domains (HBDs); and a reporter domain allows R loops to be detected and imaged in live cells. This may allow, for example, the specific and sensitive determination of the abundance, distribution and/or dynamics of R-loops in cells and may be useful for example in R-loop mapping and other applications.

A first aspect of the invention provides a method of detecting an R-loop in a cell comprising:
(i) culturing an isolated cell expressing a DNA:RNA hybrid-binding fusion protein that comprises:
   (a) two or more hybrid binding domains (HBDs); and
   (b) a detectable reporter domain,
such that the fusion protein binds to R-loops in the cell; and
(ii) determining the distribution of the fusion protein within the cell, wherein an accumulation of fusion protein is indicative of an R-loop in the cell.

A second aspect of the invention provides a DNA:RNA hybrid-binding fusion protein, comprising:
(i) two or more hybrid binding domains (HBDs); and
(ii) a reporter domain.

Preferred hybrid binding domains (HBDs) of the first and second aspects include HBDs from RNAse enzymes, such as human RNAseH1 (i.e. RNAse HBDs) and may for example comprise the amino acid sequence of SEQ ID NO: 1 or a variant thereof.

A third aspect of the invention provides a nucleic acid encoding a DNA:RNA hybrid-binding fusion protein of the second aspect.

A fourth aspect of the invention provides a vector comprising a nucleic acid of the third aspect.

A fifth aspect of the invention provides an isolated cell that expresses a DNA:RNA hybrid-binding fusion protein of the second aspect. The isolated cell may comprise a nucleic acid of the third aspect or a vector of the fourth aspect.

A sixth aspect of the invention provides a method for producing a cell that expresses a DNA:RNA hybrid-binding fusion protein, for example an isolated cell of the fifth aspect, comprising introducing a nucleic acid of the third aspect or a vector of the fourth aspect into an isolated cell.

A seventh aspect of the invention provides a method of screening for a compound that modulates R-loops comprising:
(i) culturing an isolated cell according to the fifth aspect in the presence of test compound, such that the fusion protein binds to R-loops in the cell; and:
(ii) determining the distribution of the fusion protein within the cell,
wherein a change in the distribution of the fusion protein in the cell in the presence relative to the absence of test compound is indicative that the compound modulates R-loops.

Aspects and embodiments of the invention are described in more detail below.

### BRIEF DESCRIPTION OF THE FIGURES

FIG. 1 shows imaging of R-loops in live cells. (A) Illustration of RHINO structure (left) and DNA:RNA hybrid labelling in an R-loop behind a transcribing RNA Pol II complex (right). The positions of the amino acids substituted in the RHINO mutants are indicated by red (W43A) and blue (KK59AA) asterisks. (B) Confocal microscopy images of RHINO expressed in U2OS cells together with mCherry-H2B to mark the cell nucleus and a corresponding max. intensity projection. RHINO spots throughout the nucleoplasm (highlighted in inset image) and intense labelled foci in the nucleoli are shown. (C) Comparison of RHINO and S9.6 antibody labelling of DNA:RNA hybrids. The nucleolar signals of S9.6 and RHINO are mutually exclusive (inset). (D) Expression of two RHINO mutant constructs and mCherry-histone-H2B. Nucleoli are defined with a dashed line. (E) Max. intensity projections of live U2OS cells expressing RHINO alone (left) or co-expressing mScarlet-i tagged human RNase H1 (right) and quantification of RHINO foci for each condition. (F) Quantification of RHINO foci upon use of a rapid inducible RNase H1. Single confocal plane of a HeLa cell co-expressing RHINO and the inducible RNase H1 pre-induction (top). Below, the same cell 1 h after RNase H1 induction (+TA). Quantification of RHINO foci before and 1 h after TA addition. (G) Max. intensity projections of random live U2OS cells expressing RHINO before and after 1 h incubation with Triptolide and quantification of RHINO foci number. (H) Max. intensity projections of the same live U2OS cells expressing RHINO before and after 1 h incubation with Triptolide and quantification of RHINO foci number.

FIG. 2 shows R-loops dynamics in nuclear substructures. (A) Labelling of nucleolar R-loops in live U2OS cells. Nucleoli are labelled by a red fluorescent nucleolar marker. Bright RHINO foci in several nucleoli are indicated with arrowheads. (B) Dissecting R-loop localization in nucleolar substructures. U2OS cells expressing RHINO were fixed and stained with an anti-UBF antibody marking the fibrillar centres/dense fibrillar component interface. Display of two super-resolution microscopy image sets acquired with structured illumination (top) and Airyscan 2 technology (bottom). (C) Measurements of nucleolar R-loops dynamics by FRAP on RHINO foci inside nucleoli. The fluorescence within the red circle was bleached (false colour images) and the recovery monitored over time. The curve displays the mean intensity (+/−SEM) of RHINO foci and diffuse GFP as control. (D) Labelling of Tel-R-loops in live U2OS cells co-expressing RHINO and iRFP-TRF2. Several telomeres show directly adjacent or overlapping labelling of R-loops (magnified details 1-5). Region 1 in an orthogonal 3D view demonstrates colocalization of R-loops and telomere labels. (E) Measurements of Tel-R-loops dynamics by FRAP of wild-type RHINO compared to both mutants and diffuse GFP at labelled telomeres. False colour images show the wild-type RHINO labelled cell with the bleach region (red circle). The curves display the mean intensity (+/−SD) of RHINO foci, RHINO mutants and diffuse GFP as control.

FIG. 3 shows R-loop imaging at an actively transcribed gene in live cells. (A) Graphic representation of the β-globin reporter gene for live cell imaging of transcription using the MS2 system. (B) Colocalization of mCherry-rtTA and MS2-GFP signals upon doxycycline addition in live U2OS reporter gene cells. The merge image and the intensity line scan show superimposed fluorescence signals. (C) Labelling of R-loops by RHINO in live U2OS cells at the actively transcribed β-globin reporter gene array. Fluorescence colocalization is featured in the inset merge image and intensity line scan. (D) Single confocal planes of RHINO at the β-globin reporter gene array labelled by mCherry-rtTA in 3D over time. The graph on the right shows the RHINO fluorescence intensity over time at the reporter gene locus. (E) Illustration of the IgM reporter gene containing an R-loop forming sequence (RFS) integrated as a single copy into a U2OS host cell line genome. Labelling of a single reporter gene locus by mCherry-rtTA juxtaposed to GFP-PP7 directly visualizing transcriptional activity in the merge image and the magnified inset with an intensity line scan. (F) Colocalization of RHINO and mCherry-rtTA at the site of the transcriptionally active reporter gene locus.

### DETAILED DESCRIPTION

This invention relates to the development of a genetically encoded R-loop sensor (referred to herein as “RHINO” or RNA:DNA hybrid-binding sensor). The R-loop sensor is a fusion protein selectively binds RNA:DNA hybrids and comprises multiple hybrid binding domains (HBDs) and a reporter domain. Expression of the sensor in live cells allows the imaging of R-loops within the live cells and the specific and sensitive determination of R-loop abundance, distribution and dynamics.

A DNA:RNA hybrid is a double stranded nucleic molecule that comprise an DNA strand and a complementary RNA strand. DNA-RNA hybrids form as transient intermediates in a number of physiological processes. In particular, DNA:RNA hybrids are present in R-loops in the nucleus of cells.

An R-loop is a non-canonical three-stranded nucleic acid structure that comprises a DNA:RNA hybrid and a displaced DNA strand. R-loops are usually formed transiently in cells during transcription when a nascent RNA molecule hybridizes with the template DNA strand. However, stable R-loops are associated with genetic damage and instability and may be present in the nucleus of cells, for example in the nucleolus or nucleoplasm. R-loops may also form in mitochondria in a cell.

An R-loop sensor expressed by a cell may bind to R-loops in the nucleus of the cell, for example in the nucleolus or nucleoplasm of the cell. Preferably, the R-loop sensor selectively binds to R-loops. For example, an R-loop sensor may selectively bind to the DNA:RNA hybrid structures of R-loops but may exhibit no binding or substantially no binding to other DNA:RNA hybrid structures (e.g. primers of Okasaki fragments).

The binding of the R-loop sensor to an R-loop leads to the accumulation or concentration of sensor at the site of the R-loop within the cell. Accumulations or concentrations of sensor may for example be detected as discrete foci over a non-specific diffuse background. Detection of the distribution of the expressed sensor within a cell, for example by imaging, allows accumulations or concentrations to be detected and the sites of R-loops identified. In imaging assays, accumulations or concentrations of R-loop sensor may be identified as signal foci (e.g., fluorescent foci). Signal foci may for example indicate the presence of an R-loop, a cluster of R-loops (e.g., formed at positions within the same gene), or individual R-loops of sufficient size to facilitate the binding of two or more R-loop sensors. In some embodiments, signal foci are identified as regions having a signal intensity (e.g., mean fluorescence intensity, MFI) that is at least 12-15% higher than the background, as determined by confocal microscopy.

The R-loop sensor is a fusion protein that is expressed within a cell. The fusion protein is a recombinant protein that is not found in nature or expressed in cells i.e. the fusion protein and its encoding nucleic acid are heterologous to the cell. The term “heterologous” refers to a polypeptide or nucleic acid that is foreign to a particular biological system, such as a host cell, and is not naturally present in that system. A heterologous polypeptide or nucleic acid may be introduced to a biological system by artificial means, for example using recombinant techniques. For example, heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type.

The fusion protein may comprise two or more, three or more, four or more, five or more, or six or more, seven or more, eight or more or nine or more hybrid binding domains (HBDs). Preferably, the fusion protein comprises between 3 and 9 HBDs, such as 3, 4, 5, 6, 7, 8 or 9 HBDs.

HBDs are protein domains that bind to DNA:RNA hybrids. DNA:RNA hybrid-binding may be conferred by protein structures, such as alpha-beta plaits, P-loop triphosphate hydrolase domains, nucleic acid-binding OB folds, DNA:RNA helicase domains, DEAD/DEAH box-type domains, or K-homology domains (Wang, I. Genome Res. 2018 Aug. 14 28:1405-1414). Suitable HBDs for use in an R-loop sensor may be from any DNA:RNA hybrid-binding protein that contains an HBD. Suitable DNA:RNA hybrid-binding proteins are known in the art (see for example Wang et al 2018 Genome Res. Genome Res. 2018 Aug. 14 28:1405-1414) and may include DDX5, FUS, HNRNPM, MATR3, NCL, NONO, PSPC1, RECQL, SFPQ, SRSF1, XRN1, XRN2, or RNase H1. Preferably, the HBD is an RNase hybrid binding domain (HBD) i.e. an HBD from an RNase enzyme, such as human RNase H1. Suitable RNase HBDs are well known in the art and include the HBD of an H1 RNase enzyme, such as human RNase H1 (residues 1-50 of the mature sequence of human RNase H1; Gene ID 246243; NP_001273763.1; Nowotny M. EMBO J. 2008 Apr. 9; 27(7): 1172-81).

An HBD, such as an RNase HBD, may consist of 25 to 100 amino acids, 30 to 75 amino acids, 40 to 60 amino acids, or 45 to 55 amino acids, preferably about 50 amino acids. In some preferred embodiments, an HBD may be an RNase HBD that comprises or consists of the amino acid sequence of any one of SEQ ID NOs 1, 2 or 4 or a variant thereof. A suitable HBD may be encoded by a nucleotide sequence of SEQ ID NO: 3 or SEQ ID NO: 5 or a variant thereof.

A variant of a reference amino acid sequence set out herein, such as a reference HBD sequence, reference linker sequence, reference reporter sequence, or reference fusion protein sequence, may comprise an amino acid sequence having at least 50%, at least 60%, at least 70%, at least 80%, at least 90%, at least 95%, or at least 98% sequence identity to the reference sequence. Particular amino acid sequence variants may differ from a reference sequence shown herein by insertion, addition, substitution or deletion of 1 amino acid, 2, 3, 4, 5, 6, 7, 8, 9, or 10 or more than 10 amino acids.

Preferably, an RNase HBD may comprise an amino acid sequence with a Tyr residue located at a position corresponding with Trp16 of SEQ ID NO: 1; a Lys residue located at a position corresponding with Lys32 of SEQ ID NO: 1; and a Lys residue located at a position corresponding with Lys33 of SEQ ID NO: 1. The RNase HBD may further comprise: an amino acid sequence with a Phe residue located at a position corresponding with Phe1 of SEQ ID NO: 1; a Tyr reside located at a position corresponding with Tyr2 of SEQ ID NO: 1; a Val residue located at a position corresponding with Val4 of SEQ ID NO: 1; an Arg residue at a position corresponding with Arg5 of SEQ ID NO: 1; an Arg residue at a position corresponding with Arg8 of SEQ ID NO: 1; a Phe residue at a position corresponding with Phe13 of SEQ ID NO: 1; a Cys residue located at a position corresponding with Cys19 of SEQ ID NO: 1; a Val residue located at a position corresponding with Val23 of SEQ ID NO: 1; a Phe residue located at a position corresponding with Phe31 of SEQ ID NO: 1; a Phe residue located at a position corresponding with Phe34 of SEQ ID NO: 1; and/or a Phe residue located at a position corresponding with Phe43 of SEQ ID NO: 1 (Nowotny et al. The EMBO Journal (2008) 27, 1172-1181).

Sequence similarity and identity are commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul S F. J Mol Biol. 1990 Oct. 5; 215 (3): 403-10), FASTA (which uses the method of Pearson W R and Lipman D J. Proc Natl Acad Sci USA. 1988 April; 85 (8): 2444-8), or the Smith-Waterman algorithm (Smith T F, Waterman M S. J Mol Biol. 1981 Mar. 25; 147 (1): 195-7) or the TBLASTN program, of Altschul et al. (1990) supra, generally employing default parameters. In particular, the psi-Blast algorithm (Altschul S F. Nucleic Acids Res. 1997 Sep. 1; 25 (17): 3389-402) may be used.

Sequence comparison may be made over the full-length of the relevant sequence described herein.

In some preferred embodiments, the fusion protein may comprise three RNase HBDs. For example, the fusion protein may comprise a first RNase HBD, a second RNase HBD and a third RNAse HBD. The first second and third HBDs may each comprise or consist of the amino acid sequence of SEQ ID NO: 1 or a variant thereof. In some embodiments, the second and third HBDs may be modified to remove internal translation start sites that might lead to the production of truncated fusion proteins. For example, the first HBD may comprise or consist of the amino acid sequence of SEQ ID NO: 3 or a variant thereof and may be encoded by the nucleotide sequence of SEQ ID NO: 4 or a variant thereof. The second and third HBDs may comprise or consist of the amino acid sequence of SEQ ID NO: 5 or a variant thereof and may be encoded by the nucleotide sequence of SEQ ID NO: 6 or a variant thereof. Preferably, the first, second and third RNase HBDs are arranged sequentially in the fusion protein in an N terminal to C terminal orientation.

The detectable reporter domain is a protein domain that generates a detectable signal. Preferably, the detectable signal is light. The signal generated by the reporter allows the distribution of the fusion protein within a cell to be determined and concentrations or accumulations indicative of R-loops identified.

Light generating reporters allow the distribution of fusion protein in a cell to be determined by standard live cell imaging techniques, such as fluorescent microscopy. Suitable reporter domains include fluorescent proteins and bioluminescent proteins.

Suitable fluorescent proteins are well-known in the art and include green fluorescent protein (GFP) and derivatives thereof, such as mGreenLantern; mNeonGreen; DsRed, and derivatives thereof; such as mCherry and tdTomato; flavin mononucleotide-binding fluorescent protein (FbFP) and derivatives thereof; small ultra-red fluorescent protein (smURFP); and derivatives thereof; mRuby and derivatives thereof, TagRFP and derivatives thereof; and synthetic fluorescent proteins such as mScarlet and derivatives thereof. In preferred embodiments, the fluorescent protein may be mNeonGreen. A suitable fluorescent protein may comprise or consist of the amino acid sequence of SEQ ID NO: 7 or a variant thereof, and may be encoded by the nucleotide sequence of SEQ ID NO: 8 or a variant thereof.

Suitable bioluminescent proteins are well-known in the art and include firefly luciferase, such as P. pyralis luciferase; Renilla luciferase, such as R. reniformis luciferase and photoproteins, such as aequorin. Other suitable bioluminescent proteins include NanoLuc and derivatives thereof (Hall, M. ACS Chem. Biol. 2012, 7, 11, 1848-1857; Suzuki, K. Nat Commun. 2016 7, 13718).

The RNase HBD domains and reporter domain may independently be linked directly or through a linker in a fusion protein described herein. In some embodiments, linkers may increase the solubility of the fusion protein by introducing charged and polar amino acids, thereby preventing the formation of protein aggregates, and increasing the bioavailability of the fusion protein in live cells. In addition, linkers may improve the flexibility of the fusion protein, thereby increasing its binding to R-loops (by facilitating optimal alignment of the HBDs and the DNA:RNA hybrid of the R-loop).

A suitable linker may consist of 1 to 60 amino acids, such as 1 to 5 amino acids, 5 to 10 amino acids, 10 to 15 amino acids, 15 to 20 amino acids, 20 to 25 amino acids, 25 to 30 amino acids, 30 to 35 amino acids, 35 to 40 amino acids, 45 to 50 amino acids, 50 to 55 amino acids, or 55 to 60 amino acids. Preferably, the linker consists of between 1 to 30 amino acids, for example 5 to 25 amino acids or 5 and 15 amino acids.

The two or more RNase HBDs in the fusion protein may be connected directed to each other or may be connected by linkers positioned between the HBDs. Suitable linkers may comprise or consist of the amino acid sequence of SEQ ID NO: 9 or a variant thereof and may be encoded by the nucleotide sequence of SEQ ID NO: 10 or a variant thereof.

The RNase HBDs and the reporter domain may be connected directed to each other or may be connected by a linker which is positioned between the HBDs and the reporter domain. In some embodiments, the linker between the HBDs and the reporter domain may be longer than the linkers between the HBDs, for example 5 to 15 amino acids longer, in order to minimise interference between the reporter domain (e.g., fluorescent protein) and the HBDs, therefore facilitating improved hybrid-binding and signal identification. Suitable linkers may comprise or consist of the amino acid sequence of SEQ ID NO:11 or a variant thereof and may be encoded by the nucleotide sequence of SEQ ID NO: 12 or a variant thereof.

In some preferred embodiments, a DNA:RNA hybrid-binding fusion protein suitable for use as an R-loop sensor as described herein may comprise or consist of the amino acid sequence of SEQ ID NO: 13 or a variant thereof.

Nucleic acids encoding DNA:RNA hybrid-binding fusion proteins suitable for use as R-loop sensors are provided. A nucleic acid may encode a DNA:RNA hybrid-binding fusion protein as described above. For example, a nucleic acid encoding a DNA:RNA hybrid-binding fusion protein may comprise or consist of the nucleotide sequence of SEQ ID NO: 14 or a variant thereof. In some embodiments, a nucleic acid encoding the fusion protein may comprise specific sequence modifications to the N-terminal of one or more HBDs. For example, the nucleic acid may encode an additional Val residue or an additional Met residue prior to the HBD sequence (e.g., between the linker and the HBD sequence). This may for example eliminate or substantially reduce translation from internal start sites that would lead to the production of truncated fusion proteins.

A variant of a reference nucleotide sequence set out herein, such as a reference fusion protein coding sequence may comprise a nucleotide sequence having at least 50%, at least 60%, at least 70%, at least 80%, at least 90%, at least 95%, or at least 98% sequence identity to the reference sequence. Particular nucleotide sequence variants may differ from a reference sequence shown herein by insertion, addition, substitution or deletion of 1 amino acid, 2, 3, 4, 5, 6, 7, 8, 9, or 10 or more than 10 nucleotides

Nucleic acid molecules may comprise DNA and/or RNA and may be partially or wholly synthetic. Reference to a nucleotide sequence as set out herein encompasses a DNA molecule with the specified sequence, and encompasses a RNA molecule with the specified sequence in which U is substituted for T, unless context requires otherwise.

A nucleic acid may be codon optimised for expression in a mammalian cell.

A nucleic acid encoding a DNA:RNA hybrid-binding fusion protein may be operably linked to a promoter. The promoter may be a constitutive promoter, or more preferably an inducible promoter. Suitable constitutive promoters include the human ubiquitin C promoter and the murine ribosomal protein L30 promoter. Suitable inducible promoters include chemically inducible promoters, such as a tetracycline-controlled transactivator, a lac operon or a lac repressor; temperature-inducible promoters; such as heat-shock protein-derived promoters; and optogenetically activated promoters. In preferred embodiments, the inducible promotor may be an inducible and tuneable promoter system such as a TetON3G® system or a Cumate switch system.

Suitable methods for the introduction and expression of heterologous nucleic acids into cells are well-known in the art and described in more detail below.

In some embodiments, nucleic acid encoding a fusion protein as described herein may be introduced directly into cells using gene editing techniques.

In other embodiments, nucleic acid encoding a fusion protein as described herein may be incorporated into an expression vector. Suitable vectors are well-known in the art and are described in more detail herein. Suitable vectors can be chosen or constructed, containing appropriate regulatory sequences, including promoter sequences, terminator fragments, polyadenylation sequences, enhancer sequences, marker genes and other sequences as appropriate. A suitable vector may for example include: a Kozak sequence, a 3′UTR, a cleavage site, a poly-adenylation signal, and/or a 5′UTR in addition to the coding sequence. An inducible vector may further include a specific promoter element and a co-expressed activator of transcription that binds to the promoter upon an induction signal, such as the addition of a chemical compound.

Preferably, the vector contains appropriate regulatory sequences to drive the expression of the nucleic acid in mammalian cells. A vector may also comprise sequences, such as origins of replication, promoter regions and selectable markers, which allow for its selection, expression and replication in bacterial hosts such as E. coli.

Vectors may be plasmids e.g. phagemid, or viral e.g. phage, as appropriate. The vector may be a DNA plasmid. Suitable viral vectors may include retroviral vectors, lentiviral vectors, adenoviral vectors, adeno-associated viral vectors, herpes simplex viral vectors, and chimeric viral vectors. Preferably, the nucleic acid construct is contained in a viral vector, most preferably a gamma retroviral vector or a lentiviral vector, such as a VSVg-pseudotyped lentiviral vector.

The cells may be transduced by contact with a viral particle comprising the nucleic acid. Viral particles for transduction may be produced according to known methods. For example, HEK293T cells may be transfected with plasmids encoding viral packaging and envelope elements as well as a lentiviral vector comprising the coding nucleic acid. A VSVg-pseudotyped viral vector may be produced in combination with the viral envelope glycoprotein G of the Vesicular stomatitis virus (VSVg) to produce a pseudotyped virus particle. For further details see, for example, Sambrook & Russell (2001) Molecular Cloning: a Laboratory Manual: 3rd edition, Cold Spring Harbor Laboratory Press.

Many known techniques and protocols for manipulation of nucleic acid, for example in the preparation of nucleic acid constructs, mutagenesis, sequencing, introduction of DNA into cells and gene expression, and analysis of proteins, are described in detail in Ausubel et al. (1999) 4^{th} eds., Short Protocols in Molecular Biology: A Compendium of Methods from Current Protocols in Molecular Biology, John Wiley & Sons.

Isolated cells that express a DNA:RNA hybrid-binding fusion protein as described herein are also provided. Suitable cells may comprise a nucleic acid or vector encoding a DNA:RNA hybrid-binding fusion protein as described above.

An isolated cell may express the DNA:RNA hybrid-binding fusion protein transiently or stably.

The isolated cell may be a mammalian cell, such as a human cell or rodent cell or a non-mammalian cell, for example an avian cell, an insect cell, such as a Drosophila cell, a nematode cell, such as a C. elegans cell, a piscine cell such as a zebrafish (D. rerio) cell, a reptilian cell or an amphibian cell.

Suitable mammalian cells may be from any tissue at any stage of differentiation and may include stem cells, such as haematopoietic stem cells, mesenchymal stem cells and epithelial stem cells; bone cells, such as osteoclasts, osteoblasts and osteocytes, blood cells, such as lymphocytes, neutrophils, basophiles and megakaryocytes, muscle cells, such as myocytes, cardiomyocytes and smooth muscle cells, fat cells, such as adipocytes, and brain or nerve cells, such as neurons and glial cells.

Suitable isolated cells may be derived from a cultured cell line or directly obtained from a subject.

In some embodiments, an isolated cell as described herein may display a disease phenotype. For example, the isolated cell may display a cancer phenotype (i.e. the cell may be a cancer cell) or a neurodegenerative phenotype. Suitable disease cells include cells derived from a cultured cell line or directly obtained from a subject having the disease. In some embodiments, the cancer may be a HCC, non-Hodgkin's lymphoma, Burkitt's lymphoma, multiple myeloma, chronic lymphocytic leukaemia, hairy cell leukaemia, prolymphocytic leukaemia, anal cancer, appendix cancer, bile duct cancer (i.e., cholangiocarcinoma), bladder cancer, brain tumour, breast cancer, cervical cancer, colon cancer, cancer of Unknown Primary (CUP), oesophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumour, prostate cancer, rectal cancer, skin cancer, stomach cancer, testicular cancer, throat cancer, thyroid cancer, uterine cancer, vaginal cancer, or vulvar cancer.

An isolated cell that expresses a DNA:RNA hybrid-binding fusion protein as described herein may be produced by a method comprising introducing a nucleic acid or vector encoding a DNA:RNA hybrid-binding fusion protein as described herein into an isolated cell.

The nucleic acid or vector may be introduced into the isolated cell by any convenient method. Suitable methods for introducing or incorporating a nucleic acid into an isolated cell are well-known to those skilled in the art. The nucleic acid to be inserted may be assembled within a construct or vector which contains effective regulatory elements which will drive transcription in the isolated cell. Suitable techniques for transporting the construct or vector into the isolated cell are well known in the art and include calcium phosphate transfection, DEAE-Dextran, electroporation, liposome-mediated transfection and transduction using retrovirus or other virus, e.g. vaccinia or lentivirus. For example, solid-phase transduction may be performed without selection by culture on retronectin-coated, retroviral vector-preloaded tissue culture plates.

In some embodiments, the nucleic acid may be operably linked to an inducible promoter and the method may further comprise inducing the cell to express the DNA:RNA hybrid-binding fusion protein, such that it binds to R-loops within the cell.

Also provided are methods of detecting R-loops and double stranded DNA:RNA hybrids in a cell. In some embodiments, a method for detecting an R-loop in a cell may comprise:
(i) culturing an isolated cell expressing a DNA:RNA hybrid-binding fusion protein as described herein, such that the fusion protein binds to R-loops in the cell; and:
(ii) determining the distribution of the expressed fusion protein within the cell,
wherein the distribution of the expressed fusion protein is indicative of the distribution of R-loops in the cell.

Preferably, the isolated cell is a live cell.

In some embodiments, the isolated cell may be within an in vitro organoid.

In other embodiments, a method for detecting an R-loop in a cell may comprise:
(i) expressing a DNA:RNA hybrid-binding fusion protein as described herein in one or more cells of a non-human organism, such that the fusion protein binds to R-loops in the one or more cells; and:
(ii) determining the distribution of the expressed fusion protein within the one or more cells,
wherein the distribution of the expressed fusion protein is indicative of the distribution of R-loops in the one or more cells of the non-human organism.

Suitable non-human organisms include small or naturally transparent/translucent organisms, such as the nematode C. elegans and zebrafish (D. rerio).

The distribution of the expressed fusion protein within the nucleus of the cell or cells may be determined, for example within the nucleolus or nucleoplasm. The distribution of the fusion protein may be determined by determining the amount of fusion protein that is present at different sites or locations within the cell or cells. Because the expressed fusion protein binds to R-loops, the fusion protein may accumulate or concentrate at the sites of R-loops within the cell or cells to form discrete foci i.e. a greater amount of fusion protein may be present at the sites of R-loops than at other sites. The presence of an accumulation or concentration of expressed fusion protein at a site in the cell is indicative of the presence of an R-loop at the site. Sites of fusion protein accumulation or concentration may be indicative of the presence of a cluster of nearby R-loops (e.g., formed at positions within the same gene), or individual R-loops of sufficient size to facilitate the binding of two or more fusion proteins.

The distribution of fusion protein may be determined by detecting the reporter domain in the cell or cells. For example, the reporter domain may produce a detectable signal, preferably light. The signal produced by the reporter domain at different sites in a cell may be detected. The intensity of the signal at a site in the cell may be indicative of the amount of fusion protein at the site. The intensity of the signal across the cell may be indicative of the distribution of the fusion protein within the cell. Increased signal intensity at a site relative to other sites, for example sites known not to contain R-loops, is indicative of the accumulation or concentration of fusion protein at the site of an R-loop. The amount of fusion protein at the one or more sites may be increased relative to the background amount of fusion protein in the cell. For example, sites of fusion protein accumulation or concentration may be identified as regions having a signal intensity (e.g., mean fluorescence intensity, MFI) that is at least 12-15% higher than the background signal intensity.

In some embodiments, the detectable signal may be light. The distribution of fusion protein in the cell may be determined by detecting light produced by the reporter domain, for example by imaging the cell. Suitable imaging techniques, such as fluorescent microscopy, chemiluminescence/bioluminescence and immuno-electron microscopy, are well-established in the art (Suzuki, K. Nat Commun. 2016 7, 13718; Ariotti et al., Dev Cell. 2015 Nov. 23; 35 (4): 513-25. The distribution of fusion protein may be determined by standard image analysis techniques for example using an automated tool or software. For example, signal foci indicative of concentrations or accumulations of fusion protein within a cell may be detected and counted and their intensity, size and/or shape measured, preferably in 3-dimensions.

The presence, number, location, duration and dynamics of R-loops within the cell may be determined from the distribution of the fusion protein in the cell. For example, the presence, number and/or intracellular or intranuclear location of the R-loops may be determined.

The total number of concentrations or accumulations of R-loop sensor, as indicated by foci of detectable signal, may be indicative of the prevalence of R-loops in a given cell type or tissue, or in a treated cell type or tissue relative to a control. An increase in the number of signal foci may be indicative of an increased number of R-loops. The intensity of the signal foci relative to background may be indicative of the number or size of R-loops at a given location within a cell relative to background.

The distribution of the fusion protein in the cell may be determined at a single time point, at multiple time points or continuously over a period of time. In some preferred embodiments, the distribution of the fusion protein in the cell may be determined in real-time in a live cell. Changes in the distribution of fusion protein over a time period may be indicative of changes in the R-loops in the cell. For example, the presence, number, location, duration and/or dynamics of R-loops in the cell may alter over the time period. In some embodiments, the speed of R-loop formation and resolution may be determined within the cell.

In some embodiments, a method for detecting an R-loop in a cell as described above may further comprise providing an isolated cell that expresses a DNA:RNA hybrid-binding fusion protein. A suitable isolated cell may be provided by introducing a nucleic acid or vector as described herein into an isolated cell as described herein.

Fusion proteins of the invention are also suitable for use in nucleic acid blotting (so-called “dot-blot” techniques) and in immunoprecipitation assays, each of which are well established in the art.

Methods described herein may be useful for example in screening for compounds that modulate the presence, number, location, duration and/or dynamics of DNA:RNA hybrids or R-loops in a cell. A method of screening for a compound that modulates R-loops in a cell may comprise:
(i) culturing an isolated cell expressing a DNA:RNA hybrid-binding fusion protein as described herein in the presence of test compound; and:
(ii) determining the distribution of the expressed fusion protein within the cell,
wherein a change in distribution of the fusion protein in the presence relative to the absence of test compound is indicative that the compound modulates R-loops in the cell.

In other embodiments, a method of screening for a compound that modulates R-loops in a non-human organism may comprise:
(i) expressing a DNA:RNA hybrid-binding fusion protein as described herein in one or more cells of a non-human organism treated with a test compound, such that the fusion protein binds to R-loops in the one or more cells; and:
(ii) determining the distribution of the expressed fusion protein within the one or more cells,
wherein a change in distribution of the fusion protein in the one or more cells in treated non-human organisms relative to untreated human organism is indicative that the compound modulates R-loops in the non-human organism.

A compound that modulates R-loops may alter the presence, number, location, duration and/or dynamics of R-loops within the cell or cells. For example, the compound may increase or reduce the number of R-loops in the cell, alter the location of R-loops in the cell, and/or increase or reduce the duration of R-loops in the cell. Such compounds may find clinical or experimental utility as novel therapeutic agents. For example, as agents for use in the treatment of cancer or degenerative disease. Methods of screening test compounds as disclosed herein may therefore be useful advantageous in identifying novel therapeutic agent.

An increase in the amount of fusion protein that concentrates or accumulates at a site in the cell or cells or an increase in the number of sites at which the fusion protein concentrates or accumulates in the cell or cells may be indicative that the test compound promotes or stabilises R-loops. A decrease in the amount of fusion protein that concentrates or accumulates at a site in the cell or cells or a decrease in the number of sites at which the fusion protein concentrates or accumulates may be indicative that the test compound reduces or destabilises R-loops.

The precise format of any of the screening or assay methods of the present invention may be varied by those of skill in the art using routine skill and knowledge. The skilled person is well aware of the need to employ appropriate control experiments.

A test compound may be an isolated molecule or may be comprised in a sample, mixture or extract, for example, a biological sample. Compounds which may be screened using the methods described herein may be natural or synthetic chemical compounds used in drug screening programmes. Extracts of plants, microbes or other organisms, which contain several characterised or uncharacterised components may also be used. In some embodiments, the test compound may be a pharmaceutical agent, such as a chemotherapeutic agent.

Suitable test compounds also include analogues, derivatives, variants and mimetics of any of the compounds listed above, for example compounds produced using rational drug design to provide test candidate compounds with particular molecular shape, size and charge characteristics suitable for modulating R-loops in a cell.

Combinatorial library technology provides an efficient way of testing a potentially vast number of different compounds for ability to modulate R-loops in a cell. Such libraries and their use are known in the art, for all manner of natural products, small molecules and peptides, among others. The use of peptide libraries may be preferred in certain circumstances.

The amount of test compound which may be added to an assay of the invention will normally be determined by trial and error depending upon the type of compound used. Typically, from about 0.001 nM to 1 mM or more concentrations of putative inhibitor compound may be used, for example from 0.01 nM to 100 μM, e.g. 0.1 to 50 μM, such as about 10 μM. Even a compound which has a weak effect may be a useful lead compound for further investigation and development.

A test compound identified as modulating R-loops in a cell may be investigated further.

A test compound identified as an R-loop modulator may be isolated and/or purified or alternatively, it may be synthesised using conventional techniques of recombinant expression or chemical synthesis. Furthermore, it may be manufactured and/or used in preparation, i.e. manufacture or formulation, of a composition such as a medicament, pharmaceutical composition or drug. Methods described herein may thus comprise formulating the test compound in a pharmaceutical composition with a pharmaceutically acceptable excipient, vehicle or carrier for therapeutic application.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above.

### Examples

### Experiment 1: Imaging of R-Loops in Live Cells.

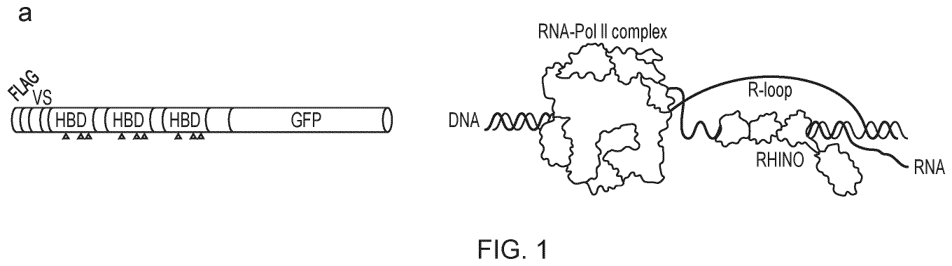

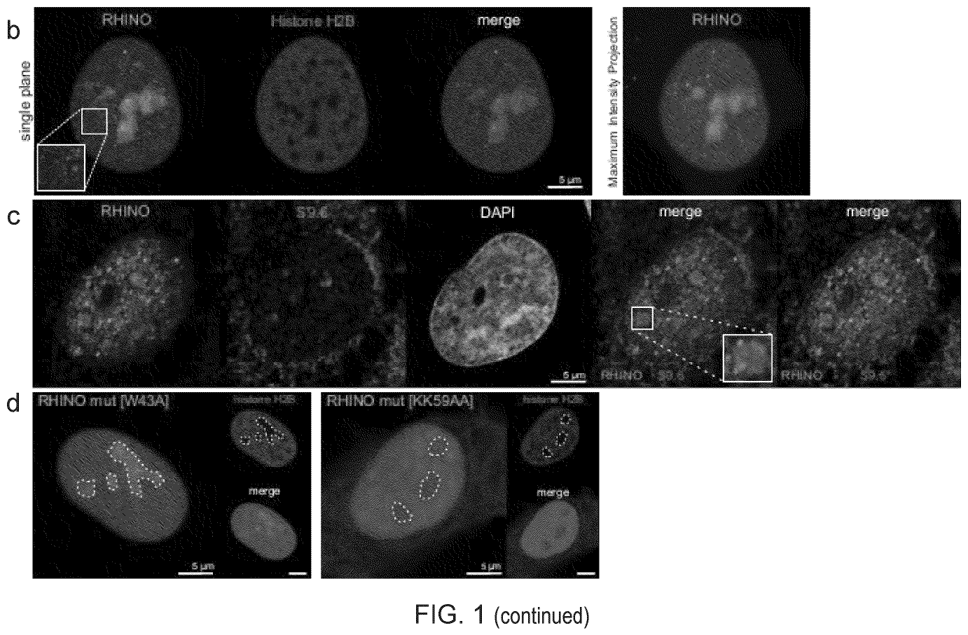

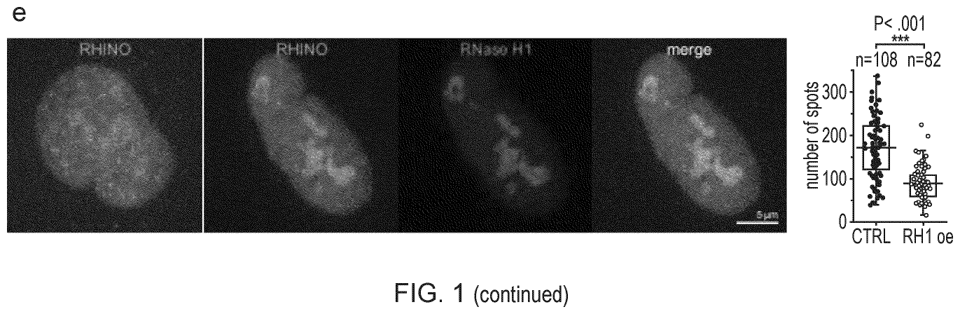

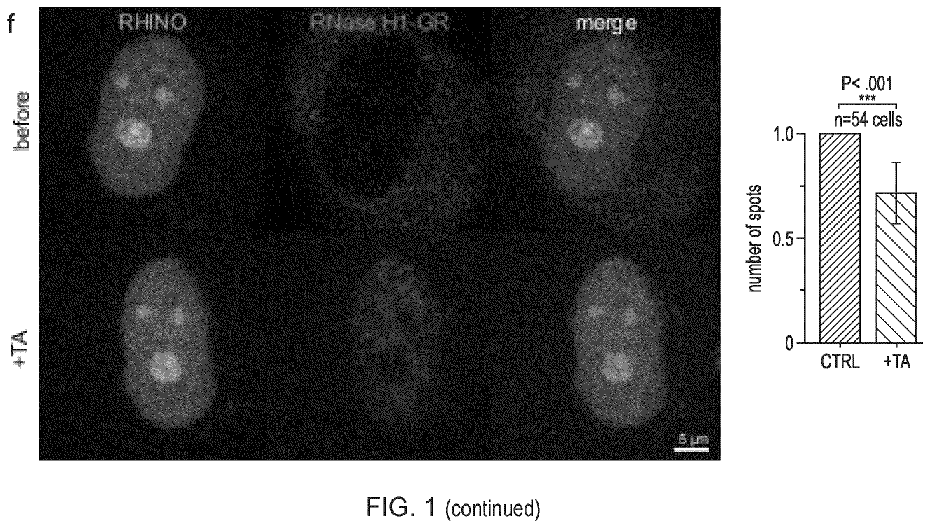

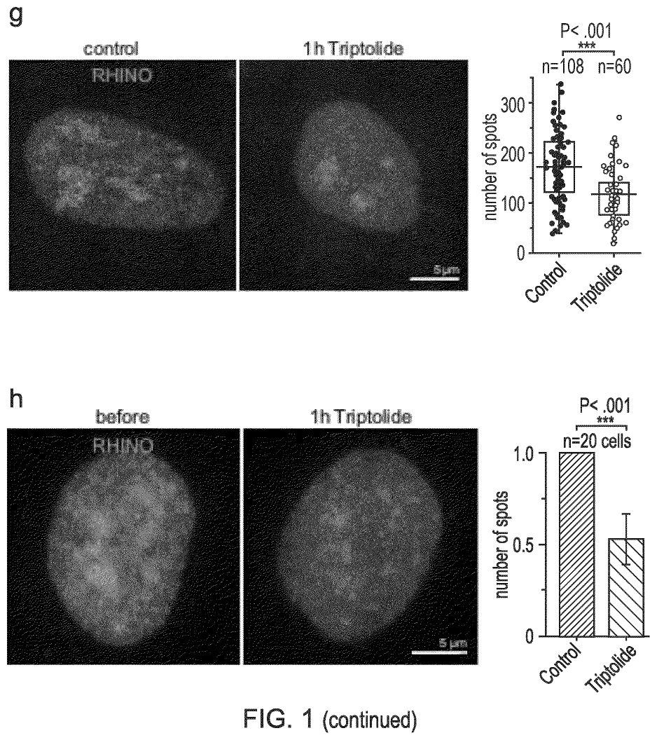

RHINO is a genetically encoded RNA:DNA hybrid-binding sensor that detects R-loops in vivo and can be used to gather novel insights into R-loops dynamics at distinct genomic locations. RHINO is a fusion protein comprising 3 HBDs and a reporter domain (e.g., a fluorescent protein) that allows real-time imaging of R-loops foci in live cells (FIG. 1A). Imaging of live human osteosarcoma (U2OS) cells transiently expressing RHINO and a fluorescent histone H2B protein (mCherry-H2B) revealed exclusively nuclear staining with a strong nucleoli definition and discrete foci of heterogeneous dimensions over a low diffuse background (inset) (FIG. 1B). Staining of RHINO-expressing U2OS cells using antibody S9.6 revealed intense cytoplasmic staining, which has been described to arise predominantly from dsRNA binding (Crossley M P. J Cell Biol. 2021 Sep. 6; 220 (9): e202101092). Differences in the RHINO and S9.6 staining profiles highlight the superior specificity of RHINO, that co-localizes with S9.6 foci in the nucleus only (FIG. 1C).

Next, two RHINO mutants were generated carrying either W43A or KK59AA amino acid substitutions on the HBD, in order to investigate the HBD structures necessary for DNA:RNA binding (Nowotny M. EMBO J. 2008 Apr. 9; 27 (7): 1172-81). Imaging U2OS cells expressing each of the two mutant sensors revealed non-specific diffuse staining covering the entire nucleus, which, in the case of the W43A mutant, included the nucleoli (FIG. 1D). The lack of discrete foci formed by any of the mutants suggests that wild type (wt) RHINO accumulates specifically at sites containing RNA:DNA hybrids.

To further evaluate the specificity of RHINO, RNase H1 was overexpressed in U2OS cells. Supporting the specific binding of RHINO to RNA:DNA hybrids, a 50% decrease in the number of R-loops foci in RNase H1-overexpressing cells was observed (FIG. 1E). A similar result was obtained upon an acute digestion of R-loops by a glucocorticoid receptor (GR)-fused RNaseH1 (RNase H1-GR) (FIG. 1F). Addition of the GR-ligand triamcinolone acetonide (TA) to cells expressing RNase H1-GR drove the translocation of RNase H1-GR into the nucleus and significantly reduced the number of R-loops foci within one hour of treatment (FIG. 1F).

As virtually all R-loops formed in the cell arise co-transcriptionally, specificity of RHINO was tested using triptolide, a global transcription inhibitor. A one-hour treatment of U2OS cells with triptolide was sufficient to significantly decrease the number of R-loops foci detected by RHINO in the bulk cell population (FIG. 1G). To follow the effect of triptolide in individual cells, the same RHINO-expressing cells were imaged before and one hour after triptolide treatment. This experiment revealed a 46% reduction in the number of R-loops foci within one hour after transcription inhibition (FIG. 1H).

### Experiment 2: Detecting R-Loops Dynamics in Nuclear Substructures.

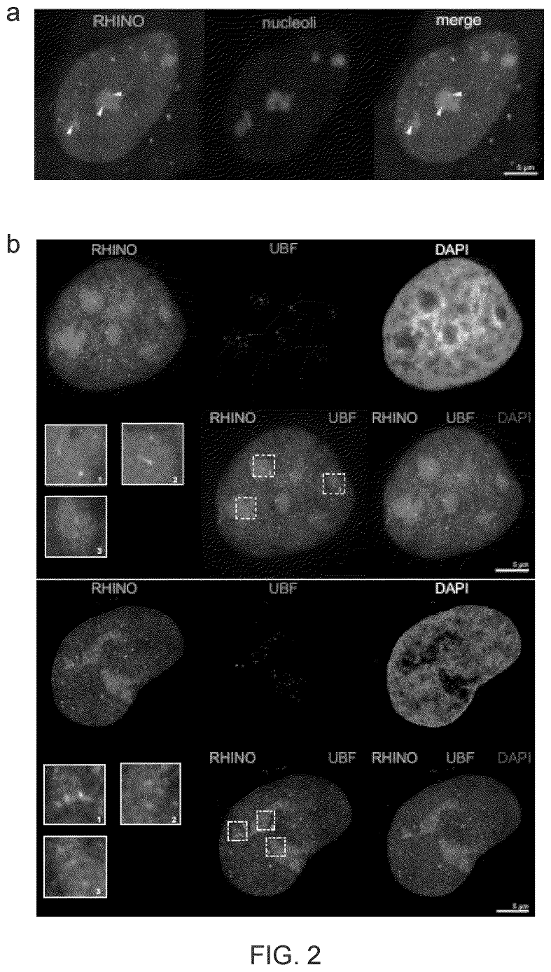

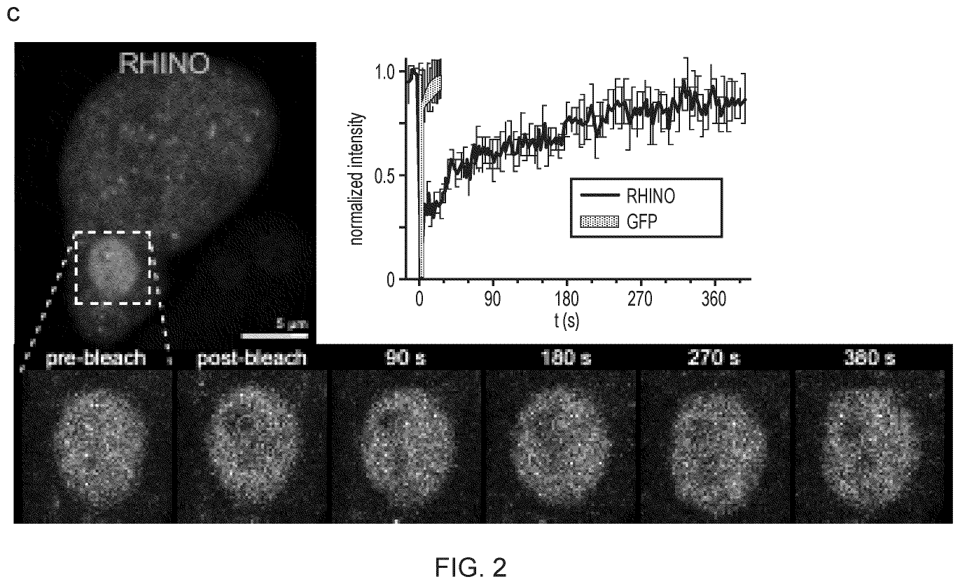

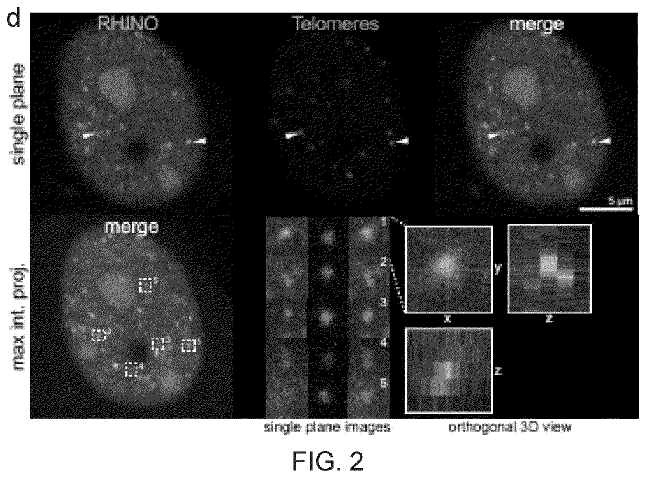

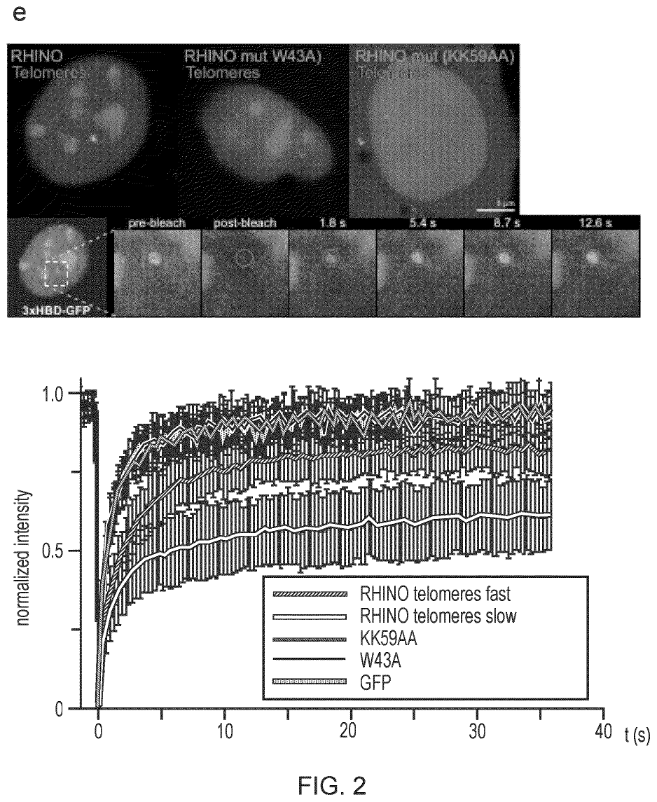

Owing to the lack of robust tools to image and collect quantitative parameters of R-loops in live cells, a clear understanding of R-loops dynamics and of their specific kinetics at distinct genomic loci is scarce. Nucleoli are nuclear structures where R-loops are abundant (Santos-Pereira J M. Nat Rev Genet. 2015 October; 16 (10): 583-97). In agreement with the findings of Experiment 1, strong nucleolar staining was observed in cells expressing RHINO, including discrete foci (FIG. 2A). Notably, the nucleolar foci co-localized with RNA Polymerase I transcription factor upstream binding factor (UBF) foci (FIG. 2B). UBF labels sites of active ribosomal RNA transcription at the periphery of the fibrillar centres and in the dense fibrillar component of nucleoli, where R-loops are abundant (Potapova, T A. Chromosome Res 27, 109-127 (2019)). To investigate the dynamics of nucleolar R-loops, fluorescence recovery was performed after photobleaching (FRAP) experiments in individual nucleolar RHINO foci (FIG. 2C). We observed a rapid turnover of fluorescent RHINO molecules, which restored 90% of the initial fluorescence intensity in 200 seconds (FIG. 2C). The RHINO kinetic profile is comparable to that of the elongation phase of RNA polymerase I on ribosomal genes, which takes up to 182 seconds to complete a transcription cycle (Dundr M. Science. 2002 Nov. 22; 298 (5598): 1623-6). Together, these data suggest that R-loops are rapidly formed and resolved at rDNA genes and illustrate the capacity of RHINO to provide kinetic parameters of these structures at specific nuclear regions in live cells.

Human cancer cells, namely telomerase-negative cells that rely on an alternative lengthening of telomeres (ALT) mechanism for telomere elongation (Sobinoff A P Trends Genet. 2017 December; 33 (12): 921-932), are characterized by high levels of telomeric R-loops (Arora R. Nat Commun. 2014 Oct. 21; 5: 5220). U2OS are amongst the ALT cells where telomeric R-loops are abundant (Bertrand E. Cell. 1998 October; 2 (4): 437-45). Several foci observed in U2OS cells expressing RHINO co-localized with TRF2 foci, a component of the shelterin complex that assembles at telomeres (FIG. 2D). Interestingly, FRAP experiments revealed two distinct populations of telomeric R-loops: a major fraction (70%) in which the fluorescence recovered to 88% of its initial intensity within 10-15 seconds after photobleaching, and another in which we little to no recovery of fluorescence was observed (FIG. 2E). FRAP experiments on cells expressing the W43A and KK59AA RHINO mutants or GFP alone, revealed a much faster fluorescence recovery that was determined by the diffusion rate of the proteins only (FIG. 2E) and suggests that the RHINO FRAP is determined by its specific binding to the RNA:DNA hybrid moiety of the telomeric R-loops.

These data disclose the existence of two classes of telomeric R-loops that differ in their dynamics: a more frequent and labile class of structures likely to correspond to co-transcriptional R-loops formed and rapidly resolved during repetitive telomeric transcription cycles, and a class of persistent R-loops that may be formed at telomeres in trans in a RAD51-dependent pathway and are not readily replaced by telomeric transcription (Feretzaki M. Nature. 2020 November; 587 (7833): 303-308).

### Experiment 3: Imaging R-Loops at an Actively Transcribed Gene in Live Cells.

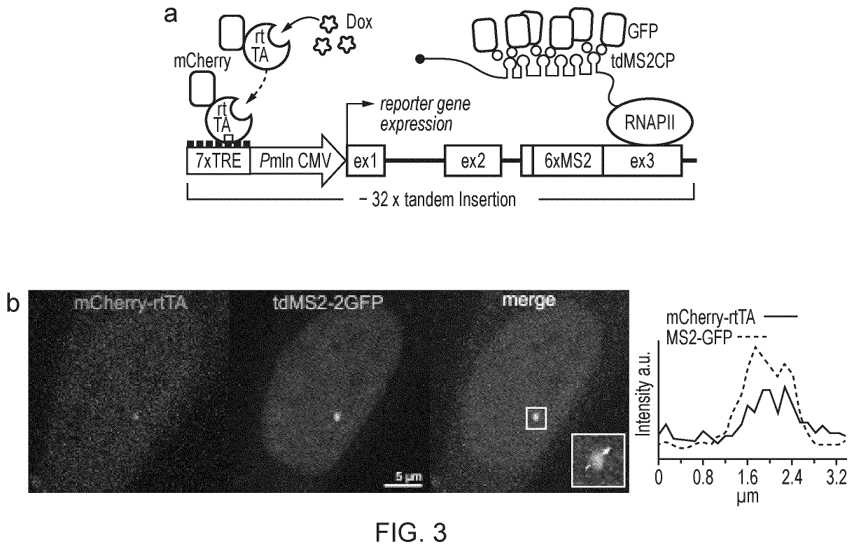

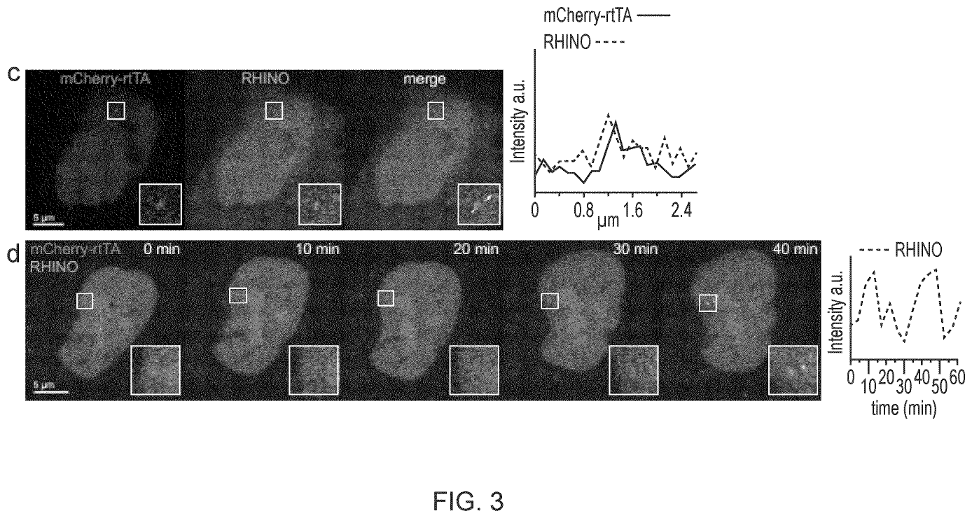

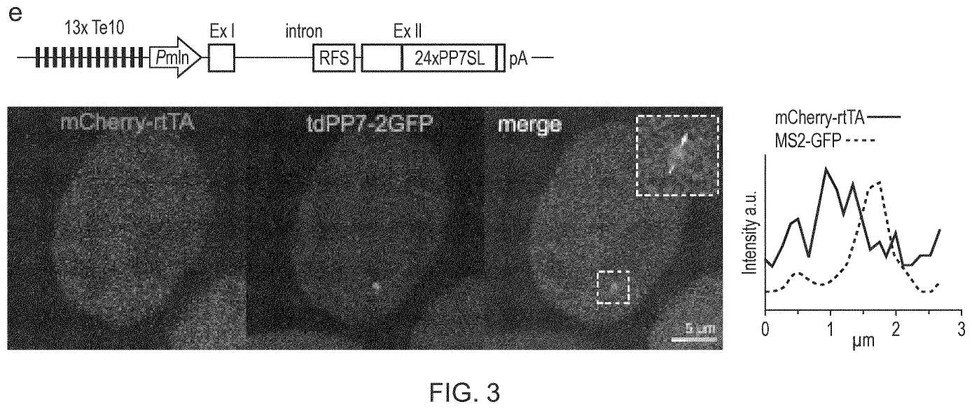

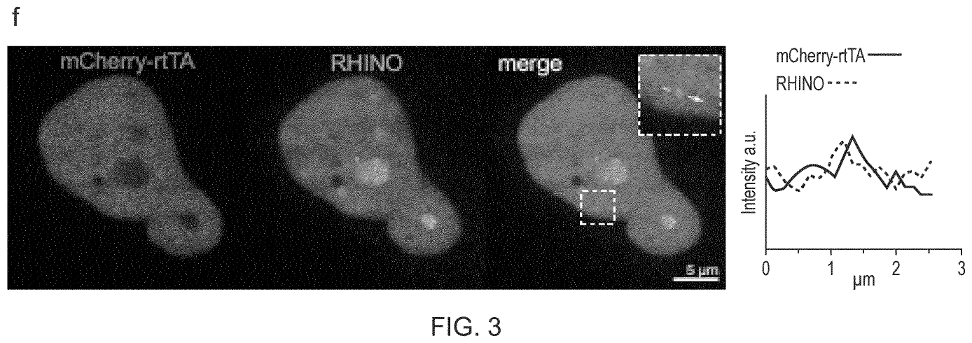

Most described physiological roles of R-loops relate to their impact on protein-coding gene transcription by RNA polymerase II (Niehrs C, Luke B. Nat Rev Mol Cell Biol. 2020 March; 21 (3): 167-178). Yet, the lack of tools to properly detect and monitor these structures in live cells precluded a deeper understanding of the mechanisms and factors governing such functions. RHINO was used to gather novel kinetic parameters of R-loops formed at protein-coding genes in human cells. A cell line containing a tandem genomic integration of a human β-globin reporter gene array carrying the MS2 system was established in order to image transcription in vivo (FIG. 3A) (Martins S B. Nat Struct Mol Biol. 2011 Sep. 4; 18 (10): 1115-23). To avoid MS2-binding protein (MS2CP) interference with R-loop formation (Bonnet A. Mol Cell. 2017 Aug. 17; 67 (4): 608-621.e6), we tagged a reverse tetracycline transactivator (rtTA) with a red fluorescent protein (mCherry) to simultaneously label the reporter gene locus and induce its transcription upon doxycycline (Dox) addition (FIG. 3A, 3B). R-loops formed at the reporter gene were imaged in live cells expressing RHINO and mCherry-rtTA, but not MS2CP, upon the addition of doxycycline (FIG. 3C). A 40-minute-long live cell imaging provided unique recordings of the real-time fluctuations of R-loop levels during consecutive transcription cycles (FIG. 3D).

To image R-loops formed during transcription of a single gene, the R-loop-prone sequence found in the β-actin gene (Skourti-Stathaki K. Mol Cell. 2011 Jun. 24; 42 (6): 794-805) was inserted within the intron of the mouse IgM reporter gene, also containing PP7 stem loops in exon II to directly visualize its transcription (FIG. 3E) (Vítor A C. Sci Adv. 2019 Jan. 9; 5 (1): eaau1249). The reporter gene locus was labelled with mCherry-rtTA. The intensity line scans show the overlap of mCherry-rtTA with PP7-GFP (FIG. 3E) and RHINO (FIG. 3F) and validates the competency of RHINO to detect R-loops formed on individual genes with high sensitivity and spatial resolution.

Together, these data demonstrate that RHINO imaging may be utilised to provide a kinetic framework for R-loop dynamics at different genomic loci: rDNA, telomeres and protein-coding genes. The ability of RHINO to collect accurate quantitative parameters from live cells affords unique opportunities for future studies seeking to delineating the reciprocal interactions between R-loops and processes such as transcription, telomere maintenance, rRNA biogenesis and other functions on which these nucleic acid structures are indicated.

### REFERENCES

Altschul S F. J Mol Biol. 1990 Oct. 5; 215 (3): 403-10.
Altschul S F. Nucleic Acids Res. 1997 Sep. 1; 25 (17): 3389-402.
Arora R. Nat Commun. 2014 Oct. 21; 5:5220
Ausubel et al. (1999) 4th eds., Short Protocols in Molecular Biology: A Compendium of Methods from Current Protocols in Molecular Biology, John Wiley & Sons.
Bertrand E. Mol Cell. 1998 October; 2 (4): 437-45.
Bou-Nader C. Nature Communications. 2022 March; 13 (1): 1641.
Bonnet A. Mol Cell. 2017 Aug. 17; 67 (4): 608-621.e6.
Cerritelli S M. Methods Mol Biol. 2022; 2528: 91-114.
Crossley M P. J Cell Biol. 2021 Sep. 6; 220 (9): e202101092.
Dundr M. Science. 2002 Nov. 22; 298 (5598): 1623-6.
Feretzaki M. Nature. 2020 November; 587 (7833): 303-308
Marnef A. Nat Cell Biol. 2021 April; 23 (4): 305-313.
Martins S B. Nat Struct Mol Biol. 2011 Sep. 4; 18 (10): 1115-23.
Niehrs C. Nat Rev Mol Cell Biol. 2020 March; 21 (3): 167-178.
Nowotny M. EMBO J. 2008 Apr. 9; 27 (7): 1172-81.
Pearson W R and Lipman D J. Proc Natl Acad Sci USA. 1988 April; 85 (8): 2444-8.
Potapova, T A. Chromosome Res 27, 109-127 (2019)
Sambrook & Russell (2001) Molecular Cloning: a Laboratory Manual: 3rd edition, Cold Spring Harbor Laboratory Press
Santos-Pereira J M. Nat. Rev Genet. 2015 October; 16 (10): 583-97
Smith T F, Waterman MS. J Mol Biol. 1981 Mar. 25; 147 (1): 195-7
Smolka J A. J Cell Biol. 2021 Jun. 7; 220 (6): e202004079.
Sobinoff A P. Trends Genet. 2017 December; 33 (12): 921-932.
Skourti-Stathaki K. Mol Cell. 2011 Jun. 24; 42 (6): 794-805
Vítor A C. Sci Adv. 2019 Jan. 9; 5 (1): eaau1249
Wang K. Sci Adv. 2021 Feb. 17; 7 (8): eabe3516.

```
Sequences
SEQ ID NO: 1
RNAse H1 HBD
FYAVRRGRKTGVFLTWNECRAQVDRFPAARFKKFATEDEAWAFVRKSAS
  SEQ ID NO: 2
RNAse H1 HBD coding sequence
TTCTATGCCGTGAGGAGGGGCCGCAAGACCGGGGTCTTTCTGACCTGGAATGAGTGCAGAGCACAGGTGGACAGATTTCC
TGCTGCCAGATTTAAGAAGTTTGCCACAGAGGATGAGGCCTGGGCCTTTGTCAGGAAATCAGCAAGC
  SEQ ID NO: 3
RNAse H1 HBD #1
MFYAVRRGRKTGVFLTWNECRAQVDRFPAARFKKFATEDEAWAFVRKSAS
  SEQ ID NO: 4
RNAse H1 HBD #1 coding sequence
ATGTTCTATGCCGTGAGGAGGGGCCGCAAGACCGGGGTCTTTCTGACCTGGAATGAGTGCAGAGCACAGGTGGACAGATT
TCCTGCTGCCAGATTTAAGAAGTTTGCCACAGAGGATGAGGCCTGGGCCTTTGTCAGGAAATCAGCAAGC
  SEQ ID NO: 5
RNAse H1 HBD #2
VFYAVRRGRKTGVFLTWNECRAQVDRFPAARFKKFATEDEAWAFVRKSAS
  SEQ ID NO: 6
RNAse H1 HBD #2 coding sequence
GTGTTCTATGCCGTGAGGAGGGGCCGCAAGACCGGGGTCTTTCTGACCTGGAATGAGTGCAGAGCACAGGTGGACAGATT
TCCTGCTGCCAGATTTAAGAAGTTTGCCACAGAGGATGAGGCCTGGGCCTTTGTCAGGAAATCAGCAAGC
  SEQ ID NO: 7
Fluorescent protein (mNeonGreen)
MVSKGEEDNMASLPATHELHIFGSINGVDFDMVGQGTGNPNDGYEELNLKSTKGDLQFSPWILVPHIGYGFHQYLPYPDG
MSPFQAAMVDGSGYQVHRTMQFEDGASLTVNYRYTYEGSHIKGEAQVKGTGFPADGPVMTNSLTAADWCRSKKTYPNDKT
IISTFKWSYTTGNGKRYRSTARTTYT FAKPMAANYLKNQPMYVFRKTELKHSKTELNFKEWQKAFTDVMGMDELYK
  SEQ ID NO: 8
Fluorescent protein (mNeonGreen) coding sequence
ATGGTGAGCAAGGGCGAGGAGGATAACATGGCCTCTCTCCCAGCGACACATGAGTTACACATCTTTGGCTCCATCAACGG
TGTGGACTTTGACATGGTGGGTCAGGGCACCGGCAATCCAAATGATGGTTATGAGGAGTTAAACCTGAAGTCCACCAAGG
GTGACCTCCAGTTCTCCCCCTGGATTCTGGTCCCTCATATCGGGTATGGCTTCCATCAGTACCTGCCCTACCCTGACGGG
ATGTCGCCTTTCCAGGCCGCCATGGTAGATGGCTCCGGCTACCAAGTCCATCGCACAATGCAGTTTGAAGATGGTGCCTC
CCTTACTGTTAACTACCGCTACACCTACGAGGGAAGCCACATCAAAGGAGAGGCCCAGGTGAAGGGGACTGGTTTCCCTG
CTGACGGTCCTGTGATGACCAACTCGCTGACCGCTGCGGACTGGTGCAGGTCGAAGAAGACTTACCCCAACGACAAAACC
ATCATCAGTACCTTTAAGTGGAGTTACACCACTGGAAATGGCAAGCGCTACCGGAGCACTGCGCGGACCACCTACACCTT
TGCCAAGCCAATGGCGGCTAACTATCTGAAGAACCAGCCGATGTACGTGTTCCGTAAGACGGAGCTCAAGCACTCCAAGA
CCGAGCTCAACTTCAAGGAGTGGCAAAAGGCCTTTACCGATGTGATGGGCATGGACGAGCTGTACAAGTAA
  SEQ ID NO: 9
Linker #1
VDGTAGPGSA
SEQ ID NO: 10
Linker #1 coding sequence
GTGGACGGAACAGCGGGCCCGGGATCTGCC
SEQ ID NO: 11
Linker #2
VDGTAGPGSTGVTGKLDPPVAT
  SEQ ID NO: 12
Linker #2 coding sequence
GTGGACGGCACCGCTGGCCCGGGATCGACCGGAGTTACAGGTAAGCTGGATCCACCGGTCGCCACC
  SEQ ID NO: 13
DNA: RNA hybrid-binding fusion protein (HBDs full underlined, spacers italicised, reporter
dashed underlined)
MFYAVRRGRKTGVFLTWNECRAQVDRFPAARFKKFATEDEAWAFVRKSASVDGTAGPGSAVFYAVRRGRKTGVFLTWNEC
RAQVDRFPAARFKKFATEDEAWAFVRKSASVDGTAGPGSAVFYAVRRGRKTGVELTWNECRAQVDRFPAARFKKFATEDE

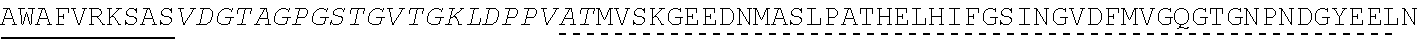

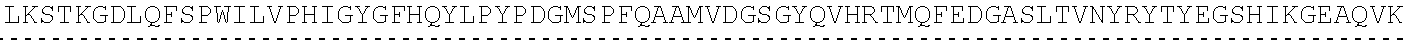

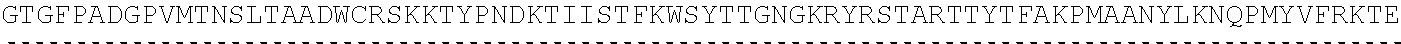

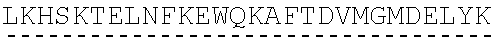




SEQ ID NO: 14
  DNA: RNA hybrid-binding fusion protein coding sequence (HBDs full underlined, spacers
italicised, reporter dashed underlined)
ATGTTCTATGCCGTGAGGAGGGGCCGCAAGACCGGGGTCTTTCTGACCTGGAATGAGTGCAGAGCACAGGTGGACAGATT
TCCTGCTGCCAGATTTAAGAAGTTTGCCACAGAGGATGAGGCCTGGGCCTTTGTCAGGAAATCAGCAAGCGTGGACGGAA
CAGCGGGCCCGGGATCTGCCGTGTTCTATGCCGTGAGGAGGGGCCGCAAGACCGGGGTCTTTCTGACCTGGAATGAGTGC
AGAGCACAGGTGGACAGATTTCCTGCTGCCAGATTTAAGAAGTTTGCCACAGAGGATGAGGCCTGGGCCTTTGTCAGGAA
ATCAGCAAGCGTGGACGGCACCGCTGGCCCGGGATCTGCCGTGTTCTATGCCGTGAGGAGGGGCCGCAAGACCGGGGTCT
TTCTGACCTGGAATGAGTGCAGAGCACAGGTGGACAGATTTCCTGCTGCCAGATTTAAGAAGTTTGCCACAGAGGATGAG
GCCTGGGCCTTTGTCAGGAAATCAGCAAGCGTGGACGGCACCGCTGGCCCGGGATCGACCGGAGTTACAGGTAAGCTGGA

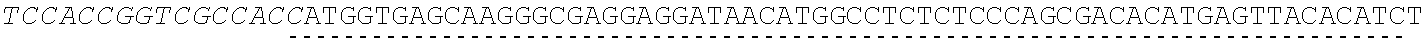

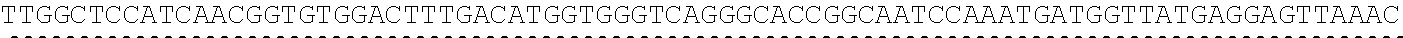

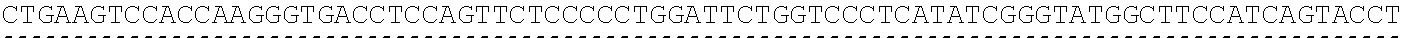

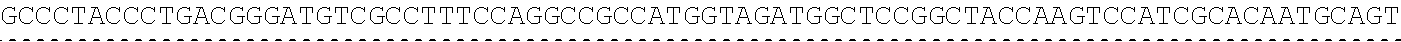

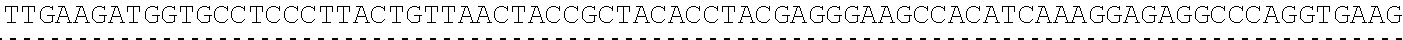

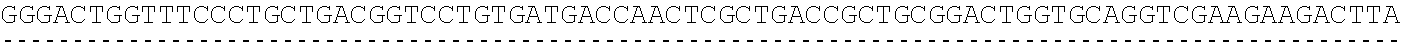

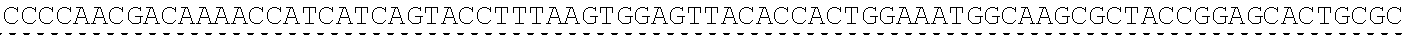

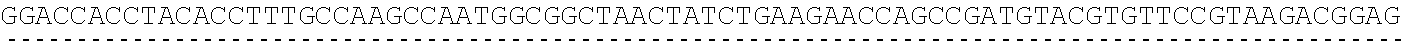

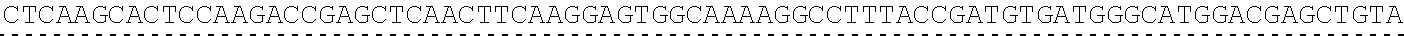

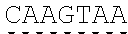










```

1. A DNA:RNA hybrid-binding fusion protein, comprising:
(i) two or more hybrid binding domains (HBDs); and
(ii) a reporter domain.

2. The fusion protein of claim 1, that binds to a double-stranded DNA:RNA hybrid.

3. The fusion protein of claim 1 or claim 2, that binds to an R-loop.

4. The fusion protein of any one of claims 1 to 3, wherein the HBD is an RNase HBD, optionally a human RNase H1 HBD, or a fragment thereof.

5. The fusion protein any one of claims 1 to 4, wherein the fusion protein comprises three HBDs.

6. The fusion protein of any one of claims 1 to 5, wherein each HBD comprises a peptide comprising a Tyr residue located at a position corresponding with Trp16 of SEQ ID NO:1; a Lys residue located at a position corresponding with Lys32 of SEQ ID NO:1; and a Lys residue located at a position corresponding with Lys33 of SEQ ID NO:1.

7. The fusion protein of any one of claim 1 to claim 6, wherein each HBD comprises or consists of the amino acid sequence set forth in SEQ ID NO:1, or a variant thereof.

8. The fusion protein of any one of claims 1 to 7, wherein the fusion protein comprises a first HBD, a second HBD and a third HBD.

9. The fusion protein of claim 8, wherein the first HBD comprises or consists of the amino acid sequence set forth in SEQ ID NO:3, or a variant thereof.

10. The fusion protein of claim 8, wherein the second and/or third HBD comprises or consists of the amino acid sequence set forth in SEQ ID NO:5, or a variant thereof.

11. The fusion protein of any one of claims 1 to 10, wherein the reporter domain comprises a fluorescent protein.

12. The fusion protein of claim 11, wherein the fluorescent protein comprises green fluorescent protein (GFP), or a derivative thereof;

13. The fusion protein of claim 12, wherein the GFP derivative is mNeonGreen.

14. The fusion protein of any one of claims 1 to 13, wherein the fusion protein further comprises one or more linker peptides.

15. The fusion protein of claim 14, comprising linker peptides positioned between the HBDs.

16. The fusion protein of claim 14 or claim 15, comprising linker peptides positioned between the HBDs and the reporter domain.

17. The fusion protein of any one of claims 14 to 16, wherein each linker peptide comprises or consists of the amino acid sequence set forth in SEQ ID NO:9, or a variant thereof.

18. The fusion protein of any one of claims 14 to 17, wherein the fusion protein comprises a first linker peptide, a second linker peptide; and a third linker peptide.

19. The fusion protein of claim 18, wherein the first and/or second linker peptides comprise or consist of the amino acid sequence set forth in SEQ ID NO:9, or a variant thereof.

20. The fusion protein of claim 18, wherein the third linker peptide comprises or consists of the amino acid sequence set forth in SEQ ID NO: 11, or a variant thereof.

21. The fusion protein of any one of claims 1 to 20, wherein the fusion protein comprises or consists of the amino acid sequence set forth in SEQ ID NO:13; or a variant thereof.

22. A nucleic acid encoding the fusion protein of any one of claims 1 to 21.

23. The nucleic acid of claim 22, comprising the nucleic acid sequence as set forth in SEQ ID NO:2, encoding a RNAse H1 HBD.

24. The nucleic acid of claim 22 or claim 23, comprising the nucleic acid sequence as set forth in SEQ ID NO: 8, encoding a fluorescent protein.

25. The nucleic acid of any one of claims 22 to 24, comprising the nucleic acid sequence as set forth in SEQ ID NO:10, encoding a linker sequence.

26. The nucleic acid of any one of claims 22 to 25, comprising or consisting of the nucleic acid sequence set forth in SEQ ID NO: 14.

27. The nucleic acid of any one of claims 22 to 26, wherein the nucleic acid is operably linked to a promoter.

28. The nucleic acid of claim 27, wherein the promoter is an inducible promoter.

29. The nucleic acid of claim 28, wherein the inducible promoter comprises a tetracycline-controlled transactivator.

30. An isolated cell, comprising the fusion protein of any one of claims 1 to 21 or the nucleic acid of any one of claims 22 to 29.

31. A method for detecting an R-loop in a cell, said method comprising:
(i) culturing an isolated cell expressing the DNA:RNA hybrid-binding fusion protein of any one of claims 1 to 21; and
(ii) identifying one or more sites in the cell at which the fusion protein accumulates, the accumulation of the fusion protein being indicative of the presence of an R-loop at the site.

32. A method of screening for a compound that modulates R-loops comprising:
(i) culturing an isolated cell expressing the DNA:RNA hybrid-binding fusion protein of any one of claims 1 to 21 in the presence of test compound, such that the fusion protein binds to R-loops in the cell; and
(ii) determining the distribution of the fusion protein within the cell;
wherein a change in the distribution of the fusion protein in the cell in the presence relative to the absence of test compound is indicative that the compound modulates R-loops.
